(19) United States
(12) Patent Application Publication
(10) Pub. No.: US 2024/0407722 A1
(43) Pub. Date: Dec. 12, 2024

(54) EAR-WORN HEARING DEVICE WITH PHYSIOLOGICAL OR ACTIVITY SENSOR

(71) Applicant: KNOWLES ELECTRONICS, LLC, ITASCA, IL (US)

(72) Inventors: CHRISTOPHER MONTI, ELGIN, IL (US); CHARLES KING, OAK PARK, IL (US); DONALD VERGHESE JACOB, CHICAGO, IL (US); JOHANNES KREUZER, MUNICH (DE); DAVID BIEGGER, MUNICH (DE); LEO LORENZ, TAUFKIRCHEN (DE)

(21) Appl. No.: 18/361,639

(22) Filed: Jul. 28, 2023

Related U.S. Application Data
(60) Provisional application No. 63/507,679, filed on Jun. 12, 2023.

(51) Int. Cl. A61B 5/00; H04R 1/10

(52) U.S. Cl. CPC A61B 5/6803; A61B 5/6817; A61B 5/6835; H04R 1/1091

(57) ABSTRACT

An ear-worn hearing device is disclose and includes a body portion with a sound-producing transducer acoustically coupled to a sound passage of a nozzle. A physiological or activity sensor is located at a side of the body portion, and a resilient lobe extends from or is disposed at least partially about the body portion. The resilient lobe has greater stiffness on a side of the body portion opposite the side at which the sensor is located, wherein the resilient lobe biases the sensor toward ear tissue when the hearing device is worn by a user.

### RELATED APPLICATIONS

This application claims priority to co-pending U.S. Provisional Patent Application Ser. No. 63/507,679, filed Jun. 12, 2023, and entitled “EAR-WORN HEARING DEVICE WITH PHYSIOLOGICAL OR ACTIVITY SENSOR”, owned by instant assignee, the entire contents of which are hereby incorporated by reference.

### FIELD OF THE DISCLOSURE

The present disclosure relates generally to ear-worn hearing devices and more particularly to ear-worn hearing devices comprising one or more physiological or activity sensors and one or more lobes that bias the sensors toward a user's ear tissue.

### BACKGROUND

Consumers have shown increasing interest in ear-worn hearing devices comprising a sensor that monitors heart rate, blood pressure, and other physiological conditions. The sensor must generally be relatively fixed near or in direct contact with ear tissue for accurate sensing. But most in-ear hearing devices tend to move within the ear during physical activity and otherwise may not optimally position the sensor for accurate sensing. To address this issue, some ear-worn hearing devices integrate the sensor with a pliable ear-tip that directly contacts ear-canal tissue. But integrating the sensor and related electronic parts with an ear-tip is complicated and costly. Additionally, ear-tips come in a variety of sizes and amplification settings to accommodate different user anatomies and varying degrees of hearing loss. Also, ear-tips are often replaced when damaged or lost. Maintaining a large inventory of, or replacing, ear-tips comprising integrated sensors further increases costs. Thus there is an ongoing need to improve ear-worn hearing devices comprising one or more sensors.

### BRIEF DESCRIPTION OF THE DRAWINGS

The objects, features and advantages of the present disclosure will become more fully apparent to those of ordinary skill in the art upon consideration of the following detailed description and appended claims in conjunction with the accompanying drawings. The drawings depict only representative embodiments and are not considered to limit the scope of the disclosure.

FIG. 1 is a representative ear-worn hearing device.

FIG. 2 a partial sectional view of a representative ear-worn hearing device.

FIG. 3 is an end view of a representative ear-worn hearing device.

FIG. 4 is a perspective view of a representative resilient lobe.

FIG. 5 is a different view of the resilient lobe of FIG. 4.

FIG. 6 is a perspective view of another representative resilient lobe.

FIG. 7 is a different view of the resilient lobe of FIG. 6.

FIG. 8 is a sectional view of FIG. 7.

FIG. 9 is partial sectional view of another representative ear-worn hearing device.

FIG. 10 is a perspective view of a first and second lobes integrated with a resilient sleeve.

FIG. 11 is a side view of FIG. 10.

FIG. 12 is an end view of FIG. 11.

FIG. 13 is a side view of FIG. 11.

FIG. 14 is a partial sectional view of another representative ear-worn hearing device.

FIG. 15 is a perspective view of the resilient lobe of FIG. 14.

FIG. 16 is a plan view of FIG. 15.

FIG. 17 is a sectional view of FIG. 16.

FIG. 18 is a sectional view of FIG. 17.

FIG. 19 is a partial sectional view of another representative ear-worn hearing device.

FIG. 20 is a sectional view of another representative ear-worn hearing device.

FIG. 21 is a sectional view of yet another representative ear-worn hearing device.

FIG. 22 is a perspective view of the resilient lobe of FIG. 20.

FIG. 23 is a plan view of FIG. 22.

FIG. 24 is an end view of FIG. 23.

FIG. 25 is a sectional view of FIG. 23.

FIG. 26 is a perspective view of a schematic representation of a resilient lobe.

FIG. 27 is a plan view of FIG. 26.

FIG. 28 is a sectional view of FIG. 27.

FIG. 29 is still another representative ear-worn hearing device.

FIG. 30 is a perspective view of the resilient lobe shown in FIG. 29.

Those of ordinary skill in the art will appreciate that the figures are illustrated for simplicity and clarity and therefore may not be drawn to scale and may not include well-known features, that the order of occurrence of actions or steps may be different than the order described, that the order of occurrence of such actions or steps may be performed concurrently unless specified otherwise, and that the terms and expressions used herein have meanings understood by those of ordinary skill in the art, except where a different meaning is specifically attributed to them herein.

### DETAILED DESCRIPTION

The present disclosure relates generally to ear-worn hearing devices and more particularly to ear-worn hearing devices comprising one or more physiological or activity sensors. The hearing device also comprises a body portion comprising a sound-producing transducer acoustically coupled to a sound passage of a nozzle. One or more resilient lobes extending from the body portion are configured to bias the one or more sensors toward the user's ear tissue (e.g., ear-canal tissue) when the hearing device is worn. The disclosure is applicable to hearing devices configured for at least partial insertion into a user's ear-canal and to hearing devices configured for wear in or on the user's concha, with or without an electrical cable assembly. Representative examples are described herein.

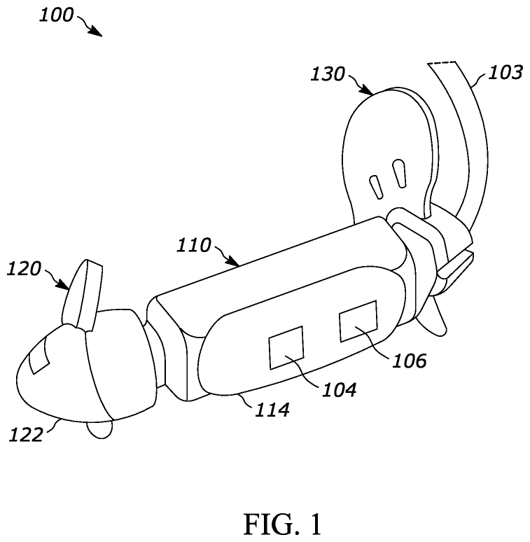

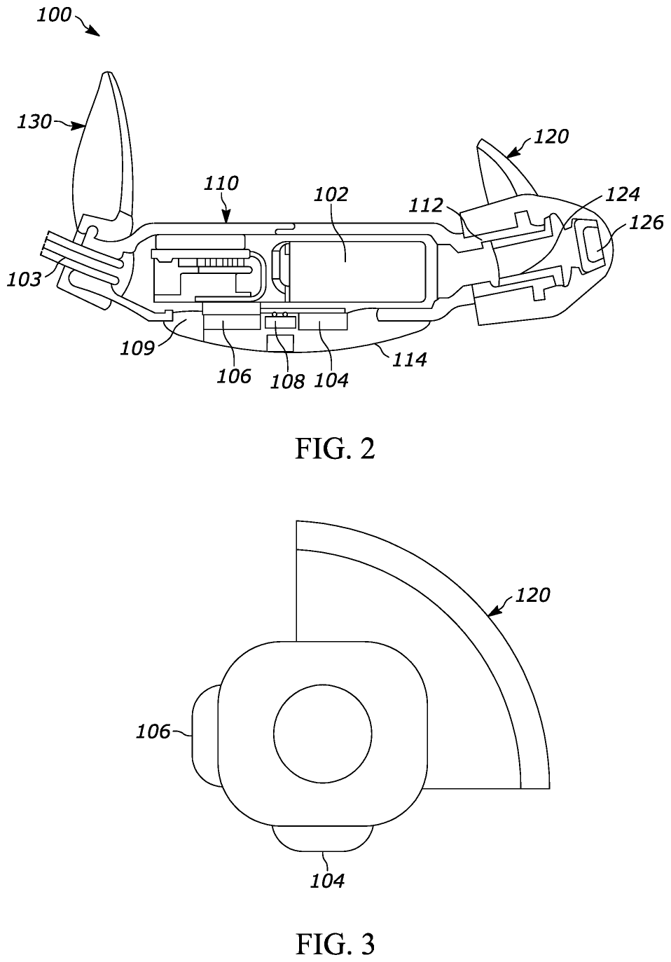

FIGS. 1, 2, 9, 14, 19-21 and 29 illustrate representative ear-worn hearing devices 100 configured for at least partial insertion into a user's ear-canal. In the representative implementations, a body portion of the hearing device comprises the sound-producing transducer 102 acoustically coupled to a sound passage of a nozzle 112. In FIGS. 2 and 19, the transducer is fully contained within a housing 110 comprising the nozzle. Alternatively, the transducer can be partially contained in an open-ended housing and the nozzle can be a spout integrated with the transducer. In this alternative, the housing can be configured as a socket into which a portion of the transducer, opposite the nozzle, is disposed and retained. Thus the body portion can comprise a housing within which the transducer is fully or partially contained. The sound-producing transducer can be implemented as one or more balanced armature receivers or dynamic speakers or a combination thereof.

Physiological sensors can monitor cardiac cycles, heart rate, blood pressure, blood oxygen, and temperature, among other physiological conditions. Representative physiological sensors include but are not limited to photoplethysmogram (PPG) sensors and temperature sensors. PPG sensors generally comprise an emitter configured as one or more single or multi-color light emitting diodes (LEDs) and a receiver configured as one or more photodiodes. Other sensors include activity sensors and electrodes for detecting various conditions. Representative activity sensors include vibration sensors and accelerometers, among others. The performance of these and other sensors can be improved when the one or more sensors contact or are in close proximity to ear tissue as described herein.

The one or more sensors are generally located at a side of the hearing device. The sensors can be mounted on a flex or other circuit board or otherwise integrated with the body portion. In FIGS. 1, 2, 9, 14, 19-21 and 29, the sensors are on a common side of the body portion. In FIG. 3, the PPG sensor parts 104 and 106 are located at a side of the body portion comprising adjacent sides or surfaces. In FIGS. 1, 2 and 19, the representative hearing devices comprises a PPG sensor including an emitter 104 and a receiver 106 and another sensor 108 implemented as a temperature, vibration or other sensor. Some hearing devices may include additional sensors. The emitter 104 and receiver 106 are integrated with the body portion and can be covered by a protective cover or lens. In FIGS. 9, 14, 20 and 21, the emitter and receiver include covers or lenses 105 and 107, respectively. In some implementations, the cover or lens functions as a light guide or pipes that transmits and directs light. The cover or lens can be configured to focus or diffuse light onto the ear tissue. A cover or lens with a glossy surface can promote wetting to the user's skin to improve light transmission into or out of the ear tissue. In FIGS. 2 and 19, the sensors are mounted on a flex circuit 109 integrated with the body portion. In FIGS. 1 and 2, the emitter 104 and receiver 106 are integrated in a convex contour 114 of the body portion. In FIG. 19, the emitter 104 and receiver 106 are integrated on a relatively flat portion of the hearing device. In either case, the one or more sensors can be biased toward the user's ear tissue when the hearing device is at least partially inserted into the ear-canal as described further herein.

The hearing device generally comprises one or more resilient lobes protruding from a side of the hearing device opposite the one or more sensors. The resilient lobe can be assembled either proximate the nozzle or proximate a portion of the housing opposite the nozzle, or at both locations. The one or more resilient lobes can be permanently or replaceably assembled with the hearing device housing. The one or more resilient lobes are flexible relative to the housing and are configured to bias the one or more sensors toward ear tissue to improve the performance of the one or more sensors upon insertion of the hearing device into the ear-canal. In FIGS. 1, 2, 9, 14 and 19, a resilient lobe 120 is assembled proximate the nozzle of the hearing device. In FIGS. 1 and 2, a second resilient lobe 130 is assembled proximate a portion of the hearing device opposite the nozzle. The one or more resilient lobes protrude from a side of the hearing device generally opposite the side at which the one or more sensors are located. In FIG. 3, the emitter 104 and receiver 106 are on adjacent sides or surface of the body portion and the resilient lobe 120 protrudes from a side portion of the hearing device generally opposite the sensors.

In FIGS. 1, 2, 9, 14, 19-21 and 29, the hearing devices comprise an electrical cable assembly 103 coupled to a portion of the hearing device opposite the nozzle. The cable assembly can connect a receiver-in-canal (RIC) or other ear-worn unit to a behind-the-ear (BTE) or other base unit. Such cable assemblies are typically shape-retaining and configured to extend between the base unit and the ear-worn unit. In some implementations, the electrical cable assembly biases the one or more sensors toward the user's ear tissue when the hearing device is worn on or at least partially in the ear. The one or more sensors can be biased toward the ear tissue by the cable assembly 103 alone or in combination with one or more resilient lobes. Other hearing devices comprising one or more sensors biased toward the user's ear tissue by one or more resilient lobes as disclosed herein are fully contained in or on the user's ear and do not required an electrical cable assembly.

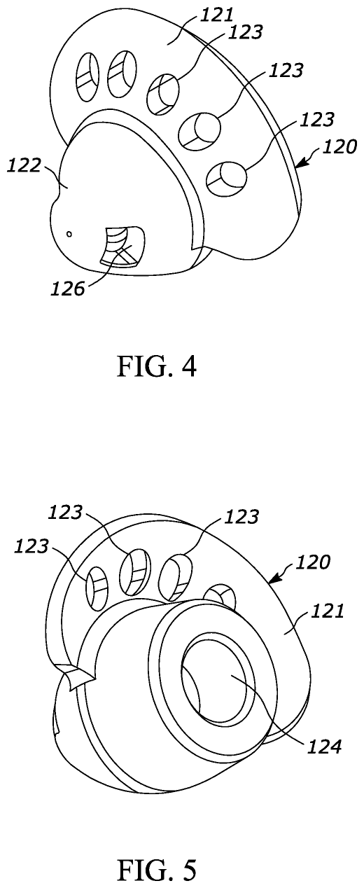

In hearing device of FIGS. 1, 2 and 9, resilient lobes 120 and 130 protrude from a side of the hearing device opposite the side at which the one or more sensors are located. In FIGS. 4 and 5, the resilient lobe 120 comprises a shell-shaped portion 121 extending from a base portion 122 having a passage 124 into which the nozzle 112 extends. The resilient nature of the base portion permits removable assembly of the resilient lobe with, and retention to, the nozzle or other portion of the hearing device. The base portion 122 can be less rigid than the shell-shaped portion 121 and can be formed from different materials using a multi-shot or insert molding operation or glued assembly among other known or future processes. The shell-shaped portion 121 can optionally comprise one or more apertures 123 to permit the passage of ambient sound into the ear-canal, to reduce occlusion effects, or to increase flexibility. Sound from the transducer is emitted from one or more sound ports 126 in the base portion 122.

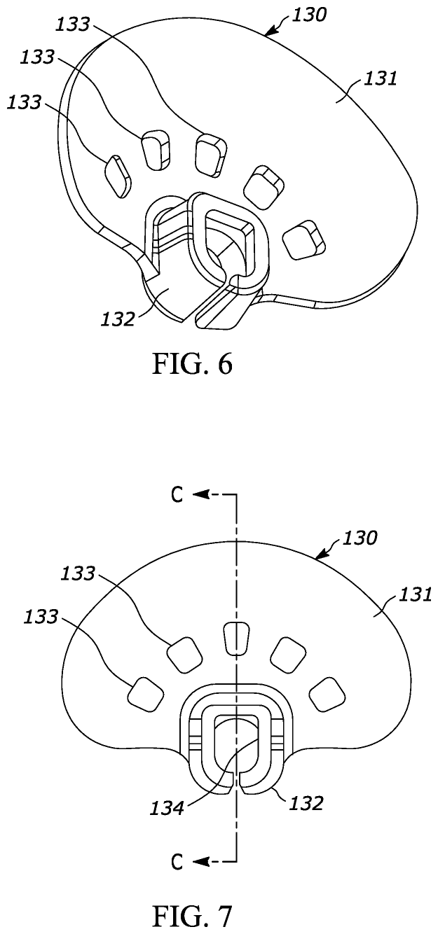

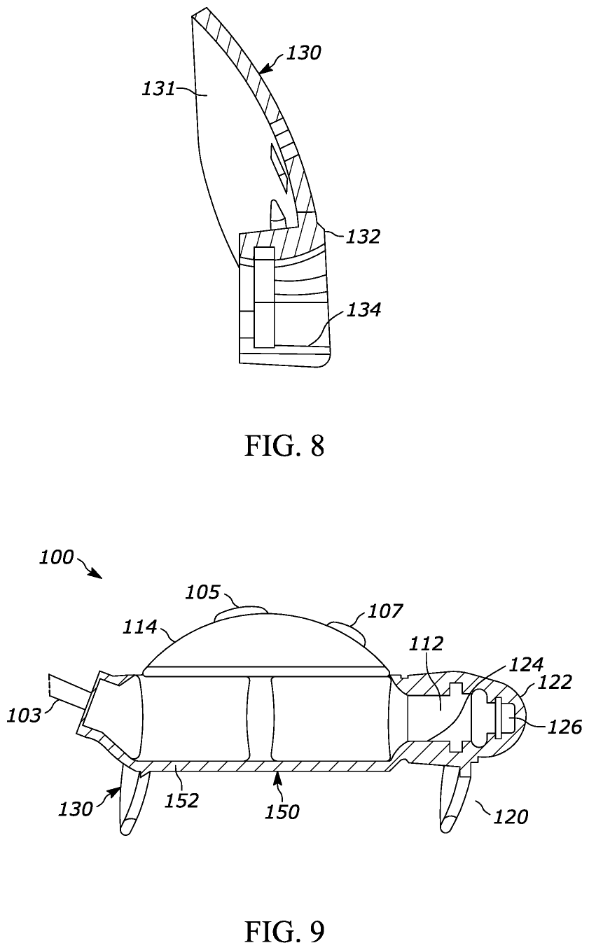

In FIGS. 6-8, the second resilient lobe 130 comprises a shell-shaped portion 131 extending from an open-ended snap-fit connector 132 defining a passage 134 that is removably assembled about an end portion of the hearing device opposite the nozzle. The resilient lobe can comprise different materials wherein the snap-fit connector 132 is more rigid than the shell-shaped portion 131. The shell-shaped portion can optionally comprise one or more apertures 133 to permit the passage of ambient sound into the ear-canal, to reduce occlusion effects, or to increase flexibility. The one or more resilient lobes can also comprise an anti-rotation feature, representative examples of which are described further herein, to maintain proper location of the one or more resilient lobes relative to the side at which the one or more sensors are located.

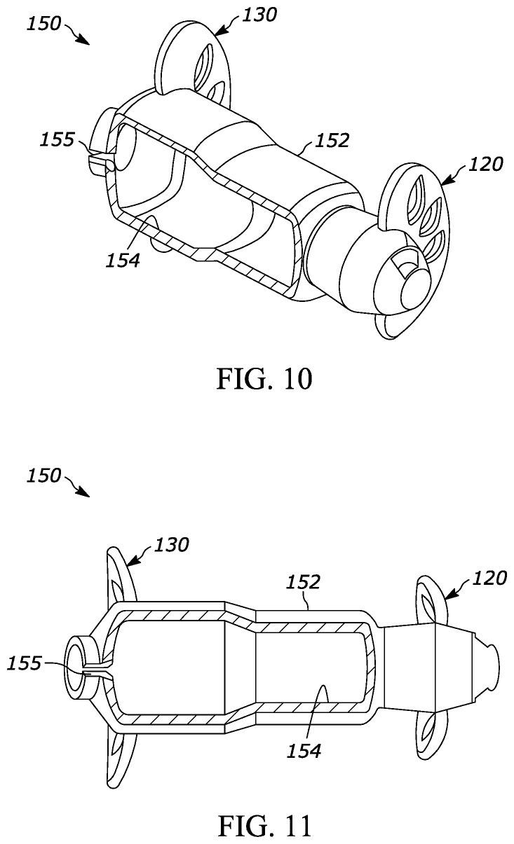

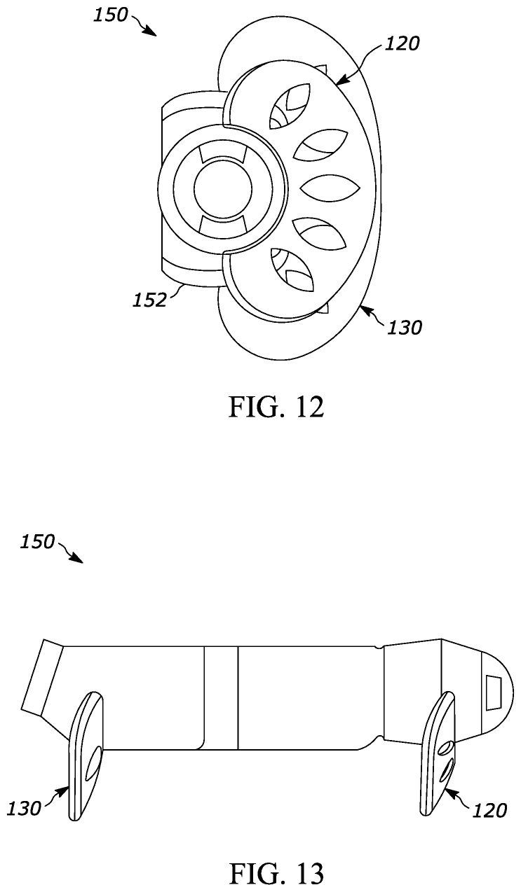

In some implementations, the one or more resilient lobes can be integrated with a resilient unitary sleeve that can be assembled at least partially about the body portion of the hearing device. The sleeve includes one or more openings to permit operation of the one or more sensors and to accommodate other structure of the body portion. The resilient nature of the sleeve permits assembly of the sleeve about the body portion. The sleeve can also include structure that properly locates the sleeve relative to other structure of the hearing device. In FIG. 9, a representative sleeve 150 is assembled about the hearing device housing and includes an opening for a convex contour 114 of the housing at which the one or more sensors are located. In FIGS. 10-13, the sleeve 150 comprises a body portion 152 from which resilient lobes 120 and 130 extend. The body portion 152 also comprises a passage into which the nozzle extends and one or more sound ports 126 from which sound from the transducer is emitted.

In FIGS. 10 and 11, a sleeve opening 154 accommodates the convex contour of the hearing device shown in FIG. 9. The opening 154 can also help locate and align the sleeve relative to the hearing device housing. In FIGS. 10 and 11, the sleeve can also comprise an opening 155 proximate the second resilient lobe 130 to accommodate a cable assembly. A portion of the sleeve including the opening 155 forms a snap-fit feature that can be removably fastened to a portion of the housing. The snap-fit feature can be more rigid that other portions of the sleeve and can comprise a different material than other portions of the sleeve as described herein.

In some implementations, the resilient lobe comprises a quasi-spheroidal surface disposed about at least a portion of the hearing device housing to bias the one or more sensors toward the user's ear tissue. The quasi-spheroidal surface has greater stiffness on the side of the hearing device opposite the side at which the one or more sensors are located, wherein the one or more sensors can be biased toward ear tissue when the hearing device is worn by the user. The greater stiffness of the quasi-spheroidal surface on one side of the hearing device can result from the quasi-spheroidal surface extending about only a portion of the hearing device housing. Alternatively, a quasi-spheroidal surface extending fully about the housing can have greater stiffness on a side of the hearing device opposite the one or more sensors due to variations in surface thickness, selection of different stiffness materials, or asymmetrically configured openings in the surface. The elastic nature of the resilient lobe enables it to be removably assembled about the hearing device housing. The quasi-spheroidal surface can also comprise an anti-rotation feature, representative examples of which are described further herein.

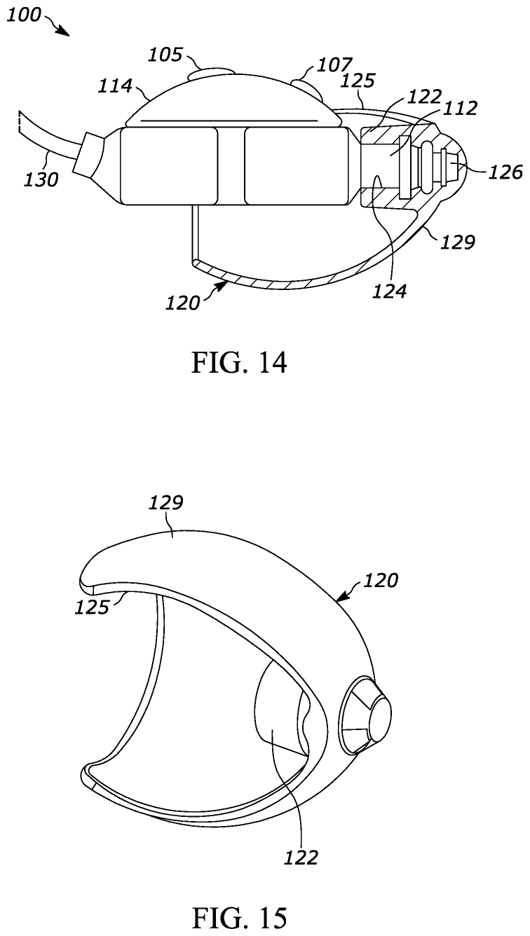

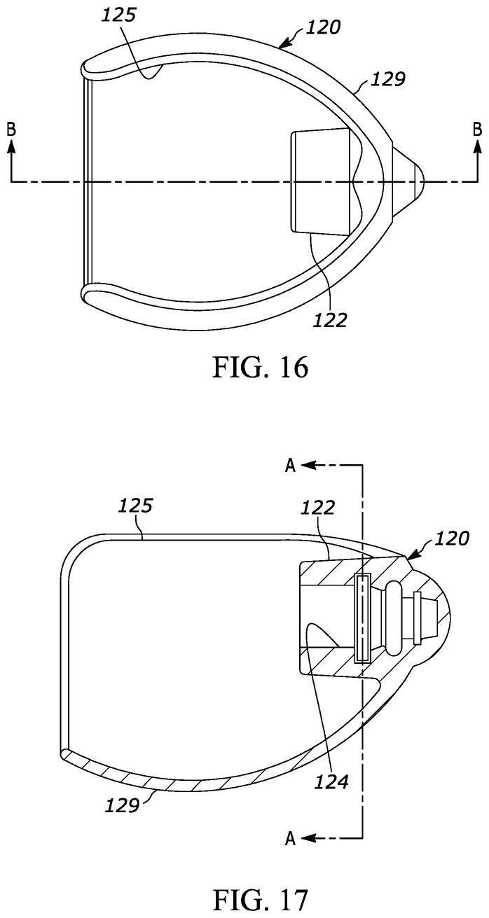

In the implementation of FIG. 14, the resilient lobe 120 comprises a quasi-spheroidal surface 129 extending from a base portion 122 having a passage 124 into which a nozzle 112 extends. The quasi-spheroidal surface comprises an opening 125 on the side of the hearing device at which the sensors 105 and 107 are located, wherein the quasi-spheroidal surface extends about only a portion of the housing opposite the sensors. Sound from the transducer is emitted from one or more sound ports 126 of the base portion 122. FIGS. 15-18 show various views of a representative resilient lobe 120 comprising the quasi-spheroidal surface 129 and opening 125 shown in FIG. 14. Thus configured, the quasi-spheroidal surface has greater stiffness on the side of the hearing device opposite the sensors and provides clearance to permit unobstructed operation of the sensors.

In the implementation of FIG. 19, the resilient lobe 120 comprises a continuous quasi-spheroidal surface 129 extending from a base portion 122 assembled proximate nozzle 112, and another end portion assembled at a portion of the hearing device opposite the nozzle as described herein with reference to FIGS. 6-8. The quasi-spheroidal surface 129 is located predominately on a side of the hearing device opposite the side on which the one or more sensors 104, 106 and 108 are located. Thus configured, the resilient lobe can bias the one or more sensors toward ear tissue when the hearing device is worn by the user. The quasi-spheroidal surface can comprise a hollow or solid structure. A vent may be optionally included.

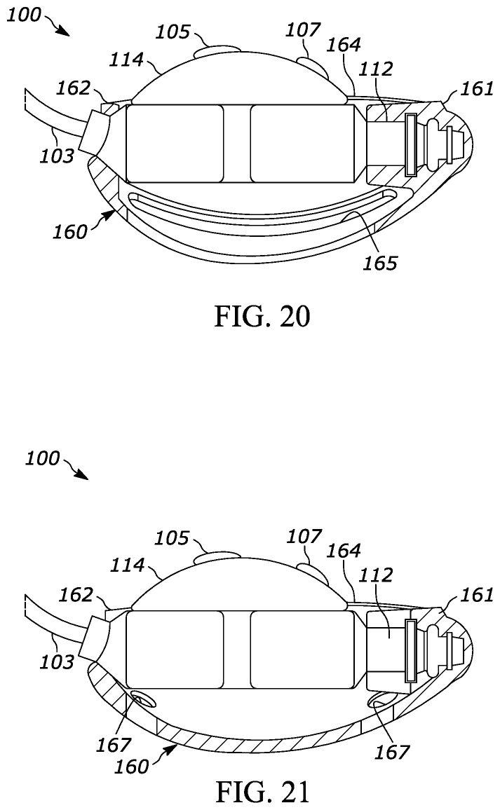

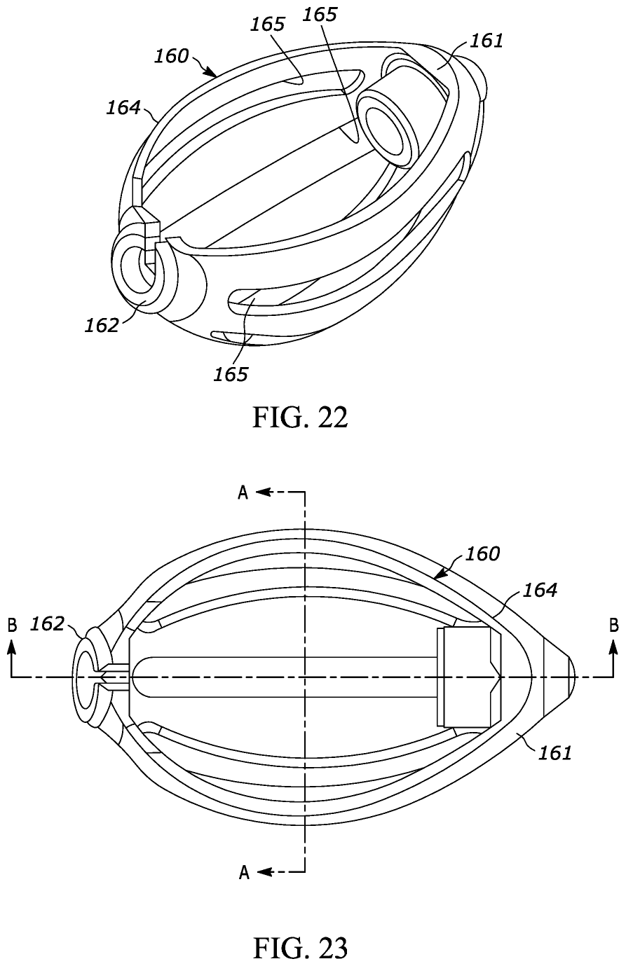

In the implementation of FIGS. 20-21, the resilient lobe comprising a quasi-spheroidal surface 160 removably assembled partially about the hearing device housing is configured to bias the one or more sensors toward the user's ear tissue. The quasi-spheroidal surface is located predominately on a side of the hearing device opposite the side on which the one or more sensors are located. The quasi-spheroidal surface comprises an opening 164 on the side of the hearing device at which the sensors are located. The quasi-spheroidal surface comprises a base portion 161 with a passage into which the nozzle 112 extends, and an open-ended snap-fit connector 162 that can be assembled at a portion of the hearing device opposite the nozzle as described herein with reference to FIGS. 6-8. In FIG. 20, the quasi-spheroidal surface 160 comprises a plurality of longitudinal openings 165 that extend along a longitudinal dimension of the hearing device when assembled therewith. FIGS. 22-25 show various views of the representative resilient lobe comprising the quasi-spheroidal surface 160 and opening 164 shown in FIG. 20. In FIG. 21, the quasi-spheroidal surface 160 comprises a plurality of openings 167. Thus configured, the quasi-spheroidal surfaces 160 of FIGS. 20 and 21 have greater stiffness on the side of the hearing device opposite the sensors and permit unobstructed operation of the sensors. The openings 165 and 167 increase flexibility of the resilient lobes for improved comfort when the hearing device is worn by the user's ear and serve as acoustic vents.

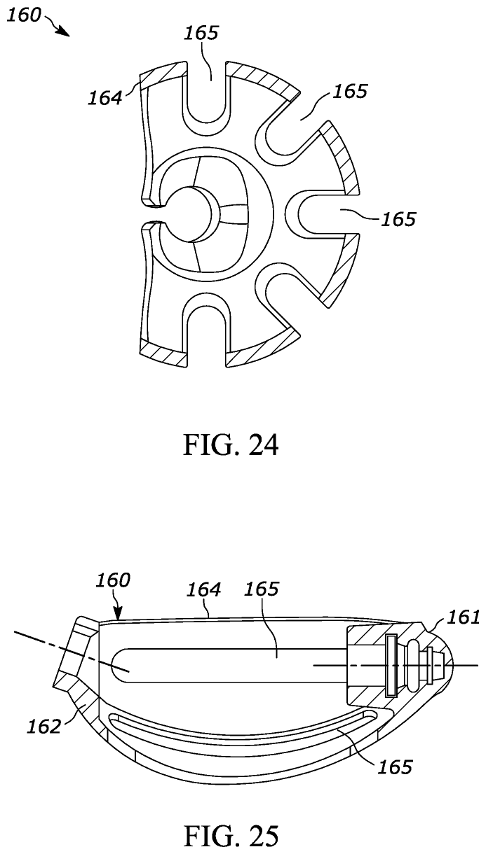

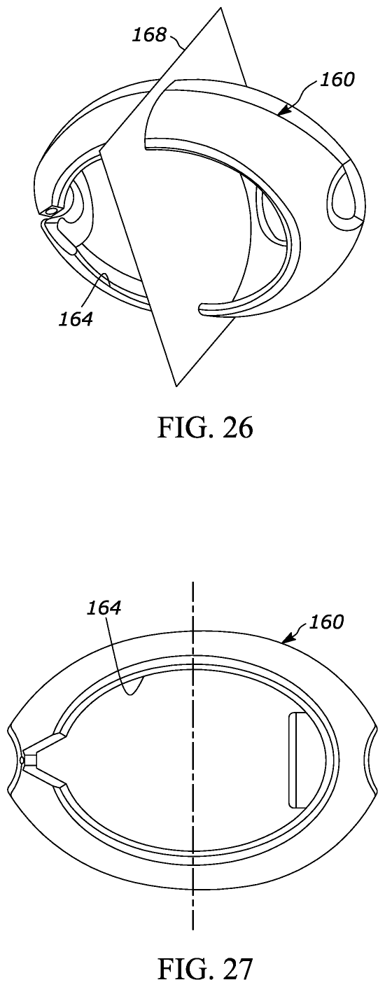

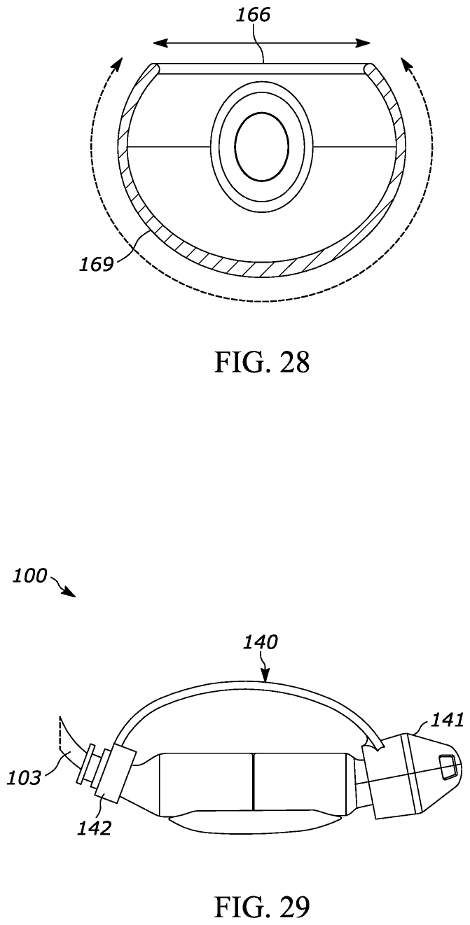

In one implementation, the opening of the quasi-spheriodal surface of FIGS. 20 and 21 is characterized by a ratio of a chord of the opening to a perimeter about the quasi-spheroidal surface (chord/perimeter) that is not less than 9 percent. The chord and perimeter are defined at a plane through the opening and transverse to a longitudinal dimension of the quasi-spheroidal surface. In FIGS. 26-28, a plane 168 is perpendicular to the longitudinal dimension of the quasi-spheroidal surface 160. In FIG. 24, the chord 166 is measured across the opening 164 and the perimeter 169 is measured about the quasi-spheroidal surface.

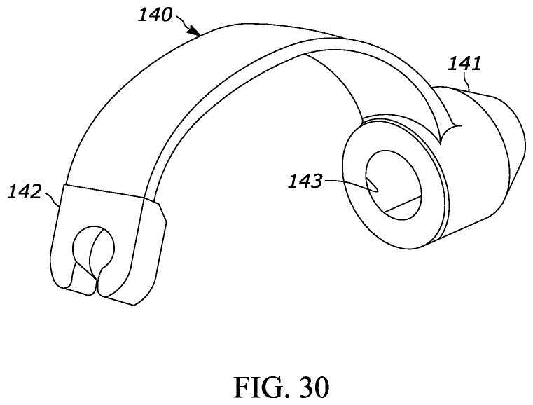

In FIGS. 29-30, the resilient lobe comprises a resilient strap 140 extending from a base portion 141 defining a passage 143 into which the nozzle extends as shown in FIG. 29. The base portion can be integrated with an ear-tip as shown in FIG. 29. Alternatively, the resilient strap can be assembled to the nozzle adjacent to a discrete ear-tip. The resilient strap 140 comprises an open-ended snap-fit connector 142 that is removably assembled at least partially about a portion of the hearing device opposite the nozzle. The resilient lobe can comprise a different material than the snap-fit connector, wherein the connector is more rigid than the strap portion as described herein. The snap-fit connector can be assembled at different locations of the housing for proper fit in the user's ear canal. The strap is located at a side of the hearing device opposite the side at which the one or more sensors are located. The resilient strap is flexible upon insertion of the hearing device at least partially into the ear-canal to bias the one or more sensors on an opposite side of the hearing device toward ear tissue. The resilient strap can also comprise an anti-rotation feature, representative examples of which are described herein.

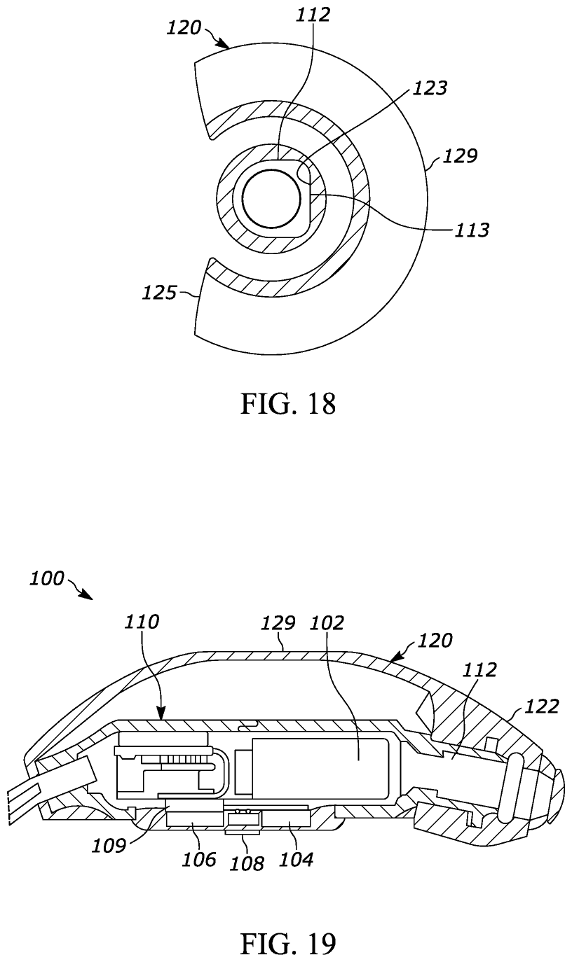

In some implementations, the hearing device comprises an anti-rotation feature to prevent rotation of the one or more resilient lobes relative to the one or more sensors. The anti-rotation feature can be located at a portion of the hearing device to which each resilient lobe is assembled. The anti-rotation feature fixes the resilient lobe to the portion of the hearing device to which the resilient lobe is assembled. The anti-rotation feature can be a keyed or irregular surface against which a complementary surface of the resilient lobe is assembled to prevent rotation of the resilient lobe relative to the nozzle or other portion of the housing to which the resilient lobe is assembled. In FIG. 7, the aperture 134 of the base portion 132 has a square or D-shaped cross-section that prevents rotation relative to a complementary portion of the hearing device to which the base portion is assembled. In FIG. 18, the cylindrical nozzle 112 comprises a flat surface 113 against which a flat surface 123 of the resilient lobe is assembled. Various other complementary surface shapes or interference fits can be employed for this purpose. An anti-rotation surface can also be formed on other portions of the hearing device where a resilient lobe is assembled. An anti-rotation feature can be implemented in any of the resilient lobes described herein.

While the disclosure and what is presently considered to be the best mode thereof has been described in a manner establishing possession and enabling those of ordinary skill in the art to make and use the same, it will be understood and appreciated that there are many equivalents to the representative embodiments described herein and that myriad modifications and variations may be made thereto without departing from the scope and spirit of the invention, which is to be limited not by the embodiments described but by the appended claims and their equivalents.

What is Claimed is:

1. An ear-worn hearing device comprising:
a body portion comprising a sound-producing transducer acoustically coupled to a sound passage of a nozzle;
a physiological or activity sensor located at a side of the body portion;
a first resilient lobe replaceably assembled to the body portion,
wherein the first resilient lobe biases the physiological sensor toward ear tissue when the hearing device is worn on or at least partially in the ear.

2. The hearing device of claim 1, the first resilient lobe protruding from a side of the body portion opposite the side at which the sensor is located, wherein the first resilient lobe is flexible relative to the body portion.

3. The hearing device of claim 2 further comprising an anti-rotation feature at a portion of the body portion to which the first resilient lobe is assembled, wherein the anti-rotation feature rotationally fixes the first resilient lobe.

4. The hearing device of claim 3, the first resilient lobe is a resilient strap assembled proximate the nozzle and proximate a portion of the hearing device opposite the nozzle.

5. The hearing device of claim 3, the first resilient lobe comprising a shell-shaped portion and assembled proximate the nozzle.

6. The hearing device of claim 3, the first resilient lobe comprising a quasi-spheroidal surface disposed about at least a portion of the body portion, wherein the quasi-spheroidal surface has greater stiffness on the side of the body portion opposite the side at which the sensor is located.

7. The hearing device of claim 6, the quasi-spheroidal surface is assembled proximate the nozzle and comprises an opening on a side of the body portion at which the sensor is located.

8. The hearing device of claim 6, the quasi-spheroidal surface comprising: a first end portion assembled proximate the nozzle; a second end portion assembled proximate a portion of the body portion opposite the nozzle; and an opening aligned with the sensor.

9. The hearing device of claim 8, the quasi-spheroidal surface comprising a plurality of longitudinal openings along a longitudinal dimension of the body portion, and an opening with which the one or more sensors are aligned.

10. The hearing device of claim 5, the quasi-spheriodal surface comprising an opening aligned with the physiological sensor, a plane through the opening and transverse to a longitudinal dimension of the quasi-spheroidal surface defining a perimeter and a chord of the quasi-spheroidal surface, a ratio of the chord to the perimeter not less than 9 percent.

11. The hearing device of claim 2, the side of the body portion at which the sensor is located comprising a convex contour, wherein the first resilient lobe biases the sensor located at the convex contour toward the ear tissue when the hearing device is worn in or at least partially in the ear.

12. The hearing device of claim 2 further comprising a second resilient lobe assembled to a portion of the body portion opposite the nozzle, the first resilient lobe assembled proximate the nozzle, wherein the second resilient lobe biases the sensor toward ear tissue when the hearing device is worn on or at least partially in the ear.

13. The hearing device of claim 1 further comprising an electrical cable assembly coupled to the body portion opposite the nozzle, wherein the electrical cable assembly biases the sensor toward the ear tissue when the hearing device is at least partially inserted into the ear-canal.

14. The hearing device of claim 13, the first resilient lobe comprises a quasi-spheroidal surface disposed about at least a portion of the hearing device, the quasi-spheroidal surface comprising: a first end portion assembled proximate the nozzle; a second end portion assembled proximate the electrical cable assembly; and an opening aligned with the sensor, wherein the quasi-spheroidal surface has greater stiffness on the side of the hearing device opposite the sensor.

15. An ear-worn hearing device comprising:
a body portion comprising a sound-producing transducer acoustically coupled to a sound passage of a nozzle;
a physiological or activity sensor located at a side of the body portion;
a resilient lobe assembled to the body portion, the resilient lobe protruding from a side of the body portion opposite the side on which the sensor is located;
anti-rotation feature at a portion of the body portion to which the resilient lobe is assembled, wherein the anti-rotation feature rotationally fixes the resilient lobe about a longitudinal dimension of the body portion,
wherein the resilient lobe biases the sensor toward ear tissue when the hearing device is worn on or at least partially in the ear.

16. The hearing device of claim 15, the resilient lobe having greater stiffness on the side of the hearing device opposite the side on which the sensor is located.

17. An ear-worn hearing device comprising:
a body portion comprising a sound-producing transducer acoustically coupled to a sound passage of a nozzle;
a physiological or activity sensor located at a side of the body portion;
a resilient lobe disposed at least partially about the body portion, the resilient lobe having greater stiffness on a side of the body portion opposite the side at which the sensor is located.

18. The hearing device of claim 17, the resilient lobe comprising a base portion assembled proximate the nozzle, a snap-fit connector assembled proximate a portion of the hearing device opposite the nozzle, and an opening aligned with the sensor.

19. The hearing device of claim 18, the resilient lobe comprising a plurality of longitudinal openings along a longitudinal dimension of the hearing device, at least one of the longitudinal openings aligned with the sensor.

20. The hearing device of claim 17, the resilient lobe comprising an opening aligned with the sensor, a plane through the opening and transverse to a longitudinal dimension of the quasi-spheroidal surface defining a perimeter and a chord of the resilient lobe, a ratio of the chord to the perimeter not less than 9 percent.
